(19) United States
(12) Patent Application Publication
(10) Pub. No.: US 2024/0238234 A1
(43) Pub. Date: Jul. 18, 2024

(54) COMPOSITION FOR PREVENTING OR TREATING OBESITY COMPRISING POLY-y-GLUTAMIC ACID ISOLATED FROM BACILLUS AS ACTIVE INGREDIENT

(71) Applicant: PUKYONG NATIONAL UNIVERSITY INDUSTRY-UNIVERSITY COOPERATION FOUNDATION, Busan (KR)

(72) Inventors: Jong-Min LEE, Busan (KR); Won-Je Jang, Busan (KR); Dong-Nyoung Oh, Busan (KR)

(73) Assignee: PUKYONG NATIONAL UNIVERSITY INDUSTRY-UNIVERSITY COOPERATION FOUNDATION, Busan (KR)

(21) Appl. No.: 18/480,145

(22) Filed: Oct. 3, 2023

(30) Foreign Application Priority Data
Jan. 6, 2023 (KR) 10-2023-0002029

(51) Int. Cl. A61K 31/198; A61P 1/14; A61P 3/04; A61P 3/08

(52) U.S. Cl. CPC A61K 31/198; A61P 1/14; A61P 3/04; A61P 3/08

(57) ABSTRACT

An object of the present disclosure is to provide a composition for preventing or treating obesity comprising a substance isolated from poly-γ-glutamic acid (γ-PGA) as an active ingredient. According to the present disclosure, it was confirmed that γ-PGAbm or γ-PGAcm, a substance isolated from γ-PGA, suppressed the expression of adipogenic marker genes in 3T3-L1 cells to reduce accumulation of lipid droplets and triglycerides, and suppressed the expression of cell cycle regulators in the early stage of adipogenesis to reduce adipocyte differentiation. In addition, γ-PGAbm or γ-PGAcm has been confirmed to suppress obesity through weight loss, positive changes in glucose and insulin resistance, reduction of epididymal adipocytes, and suppression of adipogenesis, and thus may be used effectively for related businesses.

### CROSS-REFERENCE TO RELATED APPLICATIONS

This application is based on and claims priority from Korean Patent Application No. 10-2023-0002029, filed on Jan. 6, 2023, with the Korean Intellectual Property Office, the disclosure of which is incorporated herein in its entirety by reference.

### TECHNICAL FIELD

An object of the present disclosure is to provide a composition for preventing or treating obesity including poly-γ-glutamic acid isolated from Bacillus as an active ingredient.

### BACKGROUND

Obesity is one of metabolic syndromes caused by excessive accumulation of fat due to an imbalance between energy intake and consumption. The obesity is a serious global problem affecting 25% of the world's population, and its incidence continues to increase. The obesity is also associated with health risks, such as hypertension, cardiovascular disease, diabetes type 2, stroke, arthritis, non-alcoholic fatty liver disease (NAFLD), and many types of cancer.

For obesity management, the obesity is treated through a strategy of combining a low-fat or low-saturated fat diet with a calorie-restricted diet and anti-obesity drugs. However, the calorie-restricted diet is difficult to sustain for a long period of time due to low consumer's preference and acceptance. Accordingly, the calorie-restricted diet was decreased over time. Most anti-obesity drugs, such as phentermine, orlistat, locaserin, bupropion, and liraglutide, have limited effectiveness and have side effects such as diarrhea, oil spots, and flatulence.

Accordingly, since more effective and safer obesity treatment is required, anti-obesity research on probiotics, functional foods, and dietary ingredients has been conducted. Gut microbiota is a potential factor in the pathophysiology of obesity and related metabolic disorders. The gut microbiota consists of beneficial bacteria known to protect intestinal mucosal permeability and control fat accumulation and obesity in a host by regulating absorption of dietary polysaccharides. Composition analysis of gut microbiota provides information on classical predictions of obesity and suggests a new method for anti-obesity therapy.

Since the association between the gut microbiota and obesity and obesity-related diseases may provide molecular information and potential mechanisms of this association, studies are required. Poly-γ-glutamic acid (γ-PGA) is a natural polymer and anionic polypeptide consisting of D- and L-glutamic acid units linked by amide linkages between α-amino and γ-carboxylic acid groups. The γ-PGA is mainly produced by Bacillus species and is generally known to be safe because it is biodegradable, edible, and has no toxicity to the human body and the environment. The γ-PGA is found in fermented foods in several Asian countries and has been applied in various industrial fields, such as low-temperature protectants, dye removers, metal chelators in the medical field, oil reducers, and biological purification biopolymer coagulants.

Accordingly, the present inventors examined in vitro an effect of γ-PGA treatment of the present disclosure on 3T3-L1 preadipocyte differentiation and adipogenesis-related gene expression. In addition, as a result of confirming the effects of γ-PGA treatment on changes in body weight, epididymal white adipocytes, and gut microorganisms in high-fat diet (HFD)-induced obese mice, it was confirmed that obesity was effectively controlled, and then the present disclosure was completed.

### SUMMARY

The present disclosure has been made in an effort to provide a food composition for preventing or improving obesity including poly-γ-glutamic acid (γ-PGA) as an active ingredient.

The present disclosure has also been made in an effort to provide a pharmaceutical composition for preventing or treating obesity including poly-γ-glutamic acid (γ-PGA) as an active ingredient.

The present disclosure has also been made in an effort to provide a feed composition for preventing or improving obesity including poly-γ-glutamic acid (γ-PGA) as an active ingredient.

The present disclosure has also been made in an effort to provide a composition for controlling intestinal bacteria caused by obesity including poly-γ-glutamic acid (γ-PGA) as an active ingredient.

The present disclosure has also been made in an effort to provide a method for increasing Clostridia, Clostridiales, Wolbachia, Anaplasmataceae, Alphaproteobacteria, Rickettsiales, Streptococcacceae, and Lactococcus, and decreasing Erysipelotrichia, Erysipelotrichales, Erysipelotrichaceae, Faecalibaculum, Turicibacter, Lactobacillaceae, and Lactobacillus, including treating poly-γ-glutamic acid (γ-PGA).

The present disclosure has also been made in an effort to provide a method for increasing Clostridia, Clostridiales, Caproiciproducens, Peptostreptococcaceae, Romboutsia, Clostridiaceae, and Clostridium, and decreasing Ileibacterium, Turicibacter, Lactobacillaceae, Faecalibaculum, Lactobacillus, Erysipelotrichia, Erysipelotrichales, Erysipelotrichaceae, and Erysipelotrichaceae, including treating poly-γ-glutamic acid (γ-PGA).

An exemplary embodiment of the present disclosure provides a food composition for preventing or improving obesity including poly-γ-glutamic acid (γ-PGA) as an active ingredient.

Another exemplary embodiment of the present disclosure provides a pharmaceutical composition for preventing or treating obesity including poly-γ-glutamic acid (γ-PGA) as an active ingredient.

Yet another exemplary embodiment of the present disclosure provides a feed composition for preventing or improving obesity including poly-γ-glutamic acid (γ-PGA) as an active ingredient.

Yet another exemplary embodiment of the present disclosure provides a composition for controlling intestinal bacteria caused by obesity including poly-γ-glutamic acid (γ-PGA) as an active ingredient.

Yet another exemplary embodiment of the present disclosure provides a method for increasing Clostridia, Clostridiales, Wolbachia, Anaplasmataceae, Alphaproteobacteria, Rickettsiales, Streptococcacceae, and Lactococcus, and decreasing Erysipelotrichia, Erysipelotrichales, Erysipelotrichaceae, Faecalibaculum, Turicibacter, Lactobacillaceae, and Lactobacillus, including treating poly-γ-glutamic acid (γ-PGA).

Yet another exemplary embodiment of the present disclosure provides a method for increasing Clostridia, Clostridiales, Caproiciproducens, Peptostreptococcaceae, Romboutsia, Clostridiaceae, and Clostridium, and decreasing Ileibacterium, Turicibacter, Lactobacillaceae, Faecalibaculum, Lactobacillus, Erysipelotrichia, Erysipelotrichales, Erysipelotrichaceae, and Erysipelotrichaceae, including treating poly-γ-glutamic acid (γ-PGA).

According to the exemplary embodiments of the present disclosure, it was confirmed that γ-PGAbm or γ-PGAcm, a substance isolated from γ-PGA, suppressed the expression of adipogenic marker genes in 3T3-L1 cells to reduce accumulation of lipid droplets and triglycerides, and suppressed the expression of cell cycle regulators in the early stage of adipogenesis to reduce adipocyte differentiation. In addition, γ-PGAbm or γ-PGAcm has been confirmed to suppress obesity through weight loss, positive changes in glucose, and insulin resistance, reduction of epididymal adipocytes, and suppression of adipogenesis, and thus may be used effectively for related businesses.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1A is a schematic diagram of differentiation of 3T3-L1 preadipocytes.

FIG. 1B is a diagram illustrating a timeline of animal experimental steps of the present disclosure.

FIG. 2A is a diagram illustrating a chemical structure of poly-γ-glutamic acid (γ-PGA).

FIG. 2B is a diagram illustrating a final product of poly-γ-glutamic acid (γ-PGA).

FIG. 2C is a diagram illustrating two isolated types of poly-γ glutamic acid (γ-PGA), γ-PGAcm and γ-PGAbm, through electrophoresis with SDS polyacrylamide gel.

FIG. 2D is a diagram illustrating poly-γ glutamic acid (γ-PGA) imaged through low vacuum scanning electron microscopy.

FIG. 2E is a diagram illustrating Fourier transform infrared (FT-IR) analysis of γ-PGAcm and γ-PGAbm.

FIG. 2F is a chromatogram showing poly-γ-glutamic acid (γ-PGA) produced in Bacillus sp. SJ-10.

FIG. 3A is a diagram showing cytotoxicity by concentration by treating 3T3-L1 cells with γ-PGAcm and γ-PGAbm.

FIG. 3B is a diagram showing triglyceride content (%) by treating 3T3-L1 cells with γ-PGAcm and γ-PGAbm.

FIG. 3C is a diagram showing lipid droplets stained with Oil Red O by treating 3T3-L1 cells with γ-PGAcm and γ-PGAbm.

FIG. 4A is a diagram showing the expression of genes related to fat markers by treating 3T3-L1 cells with γ-PGAcm and γ-PGAbm.

FIG. 4B is a diagram showing confirmation of fat formation for each differentiation stage by treating 3T3-L1 cells with γ-PGAbm.

FIG. 4C is a diagram showing confirmation of fat formation for each differentiation stage by treating 3T3-L1 cells with γ-PGAcm.

FIG. 4D is a diagram showing the expression levels of genes related to cell cycle regulation by treating 3T3-L1 cells with γ-PGAcm and γ-PGAbm.

FIG. 5 is a diagram showing a mechanism of poly-γ glutamic acid (γ-PGA) on adipocyte differentiation and mitotic clonal expansion (MCE).

FIG. 6A is a diagram showing changes in body weight in a normal diet (NFD), a high-fat diet (HFD), and γ-PGAbm and γ-PGAcm-supplemented groups in an obese mouse model.

FIG. 6B is a diagram showing changes in weight gain a normal diet (NFD), a high-fat diet (HFD), and γ-PGAbm and γ-PGAcm-supplemented groups in an obese mouse model.

FIG. 6C is a diagram showing diet efficiency in a normal diet (NFD), a high-fat diet (HFD), and γ-PGAbm and γ-PGAcm-supplemented groups in an obese mouse model.

FIG. 6D is a diagram showing changes in blood glucose level in a normal diet (NFD), a high-fat diet (HFD), and γ-PGAbm and γ-PGAcm-supplemented groups in an obese mouse model.

FIG. 6E is a diagram showing fasting glucose levels in a normal diet (NFD), a high-fat diet (HFD), and γ-PGAbm and γ-PGAcm-supplemented groups in an obese mouse model.

FIG. 6F is a diagram showing insulin resistance (HOMA-IR) in a normal diet (NFD), a high-fat diet (HFD), and γ-PGAbm and γ-PGAcm-supplemented groups in an obese mouse model.

FIG. 7A is a diagram showing white fat levels in a normal diet (NFD), a high-fat diet (HFD), and γ-PGAbm and γ-PGAcm-supplemented groups in an obese mouse model.

FIG. 7B is a diagram showing white fat ratios in a normal diet (NFD), a high-fat diet (HFD), and γ-PGAbm and γ-PGAcm-supplemented groups in an obese mouse model.

FIG. 7C is a diagram showing lipids stained with Oil Red O in a normal diet (NFD), a high-fat diet (HFD), and γ-PGAbm and γ-PGAcm-supplemented groups in an obese mouse model.

FIG. 7D is a diagram showing lipid area ratios in a normal diet (NFD), a high-fat diet (HFD), and γ-PGAbm and γ-PGAcm-supplemented groups in an obese mouse model.

FIG. 8A is a diagram showing the expression of Firmicutes in a normal diet (NFD), a high-fat diet (HFD), and γ-PGAbm and γ-PGAcm-supplemented groups in an obese mouse model.

FIG. 8B is a diagram showing Firmicutes/Bacteroidetes ratios in a normal diet (NFD), a high-fat diet (HFD), and γ-PGAbm and γ-PGAcm-supplemented groups in an obese mouse model.

FIG. 8C is a diagram showing genus levels of bacterial expression in a normal diet (NFD), a high-fat diet (HFD), and γ-PGAbm and γ-PGAcm-supplemented groups in an obese mouse model.

FIG. 8D is a diagram showing a principal coordinate analysis score chart of (3-diversity in a normal diet (NFD), a high-fat diet (HFD), and γ-PGAbm and γ-PGAcm-supplemented groups in an obese mouse model.

FIG. 8E is a diagram showing linear discriminant analysis effect size (LEfSe) of the differential abundance of taxa through comparison between a normal diet (NFD) and a high-fat diet (HFD).

FIG. 8F is a diagram showing linear discriminant analysis effect size (LEfSe) of the differential abundance of taxa through comparison between a γ-PGAbm-supplemented group and a high-fat diet (HFD).

FIG. 8G is a diagram showing linear discriminant analysis effect size (LEfSe) of the differential abundance of taxa through comparison between a γ-PGAcm-supplemented group and a high-fat diet (HFD).

### DETAILED DESCRIPTION

In the following detailed description, reference is made to the accompanying drawing, which forms a part hereof. The illustrative embodiments described in the detailed description, drawing, and claims are not meant to be limiting. Other embodiments may be utilized, and other changes may be made, without departing from the spirit or scope of the subject matter presented here.

Hereinafter, an exemplary embodiment of the present disclosure will be described in detail with reference to the accompanying drawings. However, the following exemplary embodiments are presented as examples for the present disclosure, and when it is determined that a detailed description of well-known technologies or configurations known to those skilled in the art may unnecessarily obscure the gist of the present disclosure, the detailed description thereof may be omitted, and the present disclosure is not limited thereto.

Terminologies used herein are terminologies used to properly express exemplary embodiments of the present disclosure, which may vary according to a user, an operator's intention, or customs in the art to which the present disclosure pertains. Therefore, these terminologies used herein will be defined based on the contents throughout the specification. Throughout the specification, unless explicitly described to the contrary, when a certain part “comprises” a certain component, it will be understood to imply the inclusion of stated elements but not the exclusion of any other elements.

All technical terms used in the present disclosure, unless otherwise defined, have the meaning as commonly understood by those skilled in the related art of the present disclosure. In addition, although preferred methods and samples are described herein, similar or equivalent methods and samples thereto are also included in the scope of the present disclosure. The contents of all publications disclosed as references in this specification are incorporated in the present disclosure.

The term “prevention” used herein may refer to all actions of suppressing or delaying the onset of obesity by administering a pharmaceutical composition for preventing or treating obesity according to the present disclosure to a subject.

The term “treatment” used herein may mean all actions which improve or benefit the symptoms of obesity by administering the composition of the present disclosure to an obesity-suspected subject.

The “improvement” used herein may mean all actions that at least reduce parameters associated with conditions to be treated, for example, the degree of symptoms.

The present disclosure provides a pharmaceutical composition for preventing or treating obesity including poly-γ-glutamic acid (γ-PGA) as an active ingredient.

In one exemplary embodiment, the poly-γ-glutamic acid (γ-PGA) may be γ-PGAbm or γ-PGAcm.

In one exemplary embodiment, the γ-PGAbm or γ-PGAcm may be isolated from Bacillus sp. SJ-10 according to a molecular weight composition.

In one exemplary embodiment, the molecular weight may be 200-1,800 kDa for γ-PGAbm and 400 kDa for γ-PGAcm.

In one exemplary embodiment, the composition suppresses differentiation of adipocytes, but is not limited thereto.

In one exemplary embodiment, the composition suppresses cell cycle regulatory genes, Cyclin-dependent kinases (CDK) 2, CDK4, cyclin A, and cyclin D, but is not limited thereto.

In one exemplary embodiment, the composition suppresses the expression of adipogenesis-related genes, but is not limited thereto.

In one exemplary embodiment, the molecular weight may be 50 kDa to 400 kDa for γ-PGAbm and 400 kDa for γ-PGAcm.

In one exemplary embodiment, the composition suppresses cell cycle regulatory genes, Cyclin-dependent kinases (CDK) 2, CDK4, cyclin A, and cyclin D, but is not limited thereto.

In one exemplary embodiment, the composition suppresses the expression of adipogenesis-related genes, but is not limited thereto.

In one exemplary embodiment, the adipogenesis-related genes are peroxisome proliferator-activated receptor gamma (PPARγ), CCAAT-enhancer-binding proteins α (C/EBPα), FAS, lipoprotein lipase (LPL), Sterol regulatory element-binding protein 1 (SREBP1), activating protein-2 (aP2), and leptin, but are not limited thereto.

In one exemplary embodiment, the composition reduces body weight, weight gain, diet efficiency, and blood glucose levels, but is not limited thereto.

In one exemplary embodiment, the composition reduces the insulin resistance and lipid content, but is not limited thereto.

In one exemplary embodiment, the composition regulates gut microorganisms, but is not limited thereto.

In one exemplary embodiment, the gut microorganisms may increase a ratio of Firmictes/Bacteroides.

In one exemplary embodiment, when γ-PGAbm is treated in the gut microorganisms, Clostridia, Clostridiales, Wolbachia, Anaplasmataceae, Alphaproteobacteria, Rickettsiales, Streptococcacceae, and Lactococcus may be increased, and Erysipelotrichia, Erysipelotrichales, Erysipelotrichaceae, Faecalibaculum, Turicibacter, Lactobacillaceae, and Lactobacillus may be decreased, thereby controlling obesity.

In one exemplary embodiment, when γ-PGAcm is treated in the gut microorganisms, Clostridia, Clostridiales, Caproiciproducens, Peptostreptococcaceae, Romboutsia, Clostridiaceae, and Clostridium may be increased, and Ileibacterium, Turicibacter, Lactobacillaceae, Faecalibaculum, Lactobacillus, Erysipelotrichia, Erysipelotrichales, Erysipelotrichaceae, and Erysipelotrichaceae may be decreased, thereby controlling obesity.

In one exemplary embodiment, the present disclosure provides a food composition for preventing or improving obesity including poly-γ-glutamic acid (γ-PGA) as an active ingredient.

In one exemplary embodiment, the present disclosure provides a feed composition for preventing or improving obesity including poly-γ-glutamic acid (γ-PGA) as an active ingredient.

In one exemplary embodiment, the present disclosure provides a composition for controlling intestinal bacteria caused by obesity including poly-γ-glutamic acid (γ-PGA) as an active ingredient.

In one exemplary embodiment, the present disclosure provides a method for increasing Clostridia, Clostridiales, Wolbachia, Anaplasmataceae, Alphaproteobacteria, Rickettsiales, Streptococcacceae, and Lactococcus, and decreasing Erysipelotrichia, Erysipelotrichales, Erysipelotrichaceae, Faecalibaculum, Turicibacter, Lactobacillaceae, and Lactobacillus, including treating poly-γ-glutamic acid (γ-PGA).

In one exemplary embodiment, the present disclosure provides a method for increasing Clostridia, Clostridiales, Caproiciproducens, Peptostreptococcaceae, Romboutsia, Clostridiaceae, and Clostridium, and decreasing Ileibacterium, Turicibacter, Lactobacillaceae, Faecalibaculum, Lactobacillus, Erysipelotrichia, Erysipelotrichales, Erysipelotrichaceae, and Erysipelotrichaceae, including treating poly-γ-glutamic acid (γ-PGA).

The adipogenesis-related genes may be PPARγ, C/EBPα, FAS, LPL, SREBP1, aP2, and leptin.

In one exemplary embodiment, the composition reduces body weight, weight gain, diet efficiency, and blood glucose levels, but is not limited thereto.

In one exemplary embodiment, the composition reduces the insulin resistance (HOMA-IR), but is not limited thereto.

In one exemplary embodiment, the composition reduces the lipid content, but is not limited thereto.

In one exemplary embodiment, the present disclosure provides a feed composition for preventing or improving obesity including poly-γ-glutamic acid (γ-PGA) as an active ingredient.

Hereinafter, the present disclosure will be described in more detail with reference to Examples. However, the following Examples are only examples to easily explain the content and scope of the technical idea of the present disclosure, and the technical scope of the present disclosure is not limited or changed thereby. In addition, based on these Examples, it may be easily determined by those skilled in the art that various modifications and changes are possible within the scope of the technical idea of the present disclosure.

### Experimental Example 1. Isolation and Characterization of Two Types of γ-PGA

Two types of γ-PGA were isolated from Bacillus sp. according to a molecular weight composition (wide range of molecular weights, γ-PGAbm; constant molecular weight, γ-PGAcm).

In a γ-PGA isolation method, Bacillus sp. SJ-10 was placed in a γ-PGA production medium (30 g/L glucose, 10 g/L NH₄Cl, 0.5 g/L K₂HPO₄, 0.5 g/L MgSO₄·7H₂O, 0.15 g/L CaCl₂·2H₂O, 0.15 g/L MgSO₄·4H₂O, and 0.04 g/L FeCl₃·7H₂O), added with a medium for 5 days, and centrifuged to remove cells. γ-PGA was precipitated by adding 3 times the volume of ethanol to the culture medium. Each protein and trace element in the precipitate were removed by boiling or dialysis. For isolation of γ-PGAcm, a γ-PGA production medium added with 8% NaCl was used, and the subsequent processes were the same. The isolated γ-PGA was characterized by performing sodium dodecyl sulfate-polyacrylamide gel electrophoresis (SDS-PAGE) and Fourier transform infrared (FT-IR) analysis and imaged through low-vacuum scanning electron microscopy (LV-SEM, JSM-6490LV & MONO CL3+). The enantiomeric composition of amino acids constituting PGA was evaluated by a method previously reported in [International journal of biological macromolecules 108 (2018) 598-607_J.M. Lee].

### Experimental Example 2. Culture and Differentiation of Preadipocytes

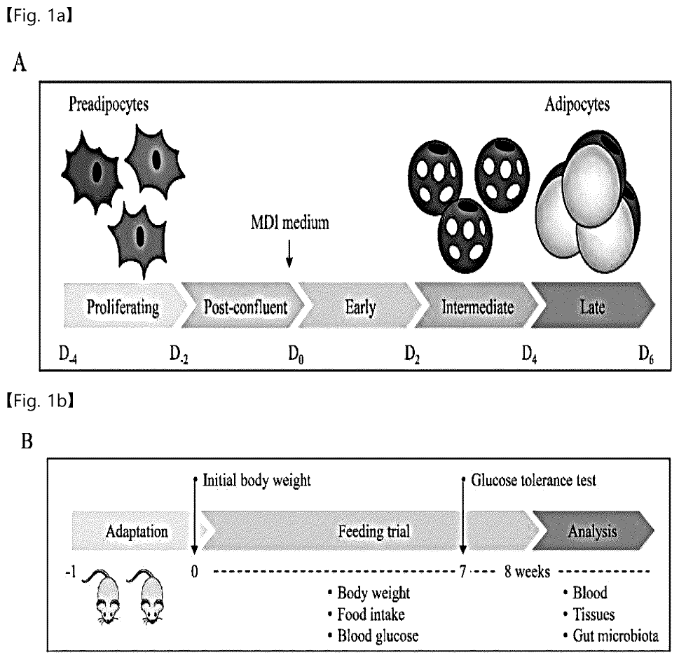

3T3-L1 preadipocytes were purchased from the Korean Cell Line Bank (Seoul, Korea) and cultured in a Dulbecco's Modified Eagle Medium (DMEM) containing 10% (v/v) fetal bovine serum (FBS) and 1% penicillin-streptomycin at 37° C. in air (5% CO₂/95% O₂). The cells were cultured in each well until 100% confluence and cultured for 48 hours to obtain fully differentiated adipocytes. After confluence, 3T3-L1 cells were induced to differentiate in an MDI medium (DMEM containing 10% FBS, 0.5 mM IBMX, 1 μM dexamethasone, and 1 μg/mL insulin) (FIG. 1A).

### Experimental Example 3. Cell Viability Assay

The viability of 3T3-L1 adipocytes was determined using MTT assay. The 3T3-L1 adipocytes were seeded in a 12-well plate and cultured until reaching a saturation state. γ-PGA was treated at a different concentration per well. After 48 hours, the adipocytes were added with an MTT solution (5 mg/ml) and incubated at 37° C. for 2 hours. Thereafter, the supernatant was removed, formazan was eluted with dimethyl sulfoxide, and the absorbance was measured at 570 nm using a microplate reader. Cell viability was expressed as percentage to a control group.

### Experimental Example 4. Oil Red O Staining of Lipid Droplets

Oil Red O staining was performed to stain and identify lipid droplet portions. Differentiation-induced cells were washed three times with phosphate buffer saline (PBS), immobilized with 3.7% formalin at room temperature for 20 minutes, and then washed with 60% isopropanol. After washing, the cells were treated with an Oil Red O solution for 20 minutes at room temperature and stained. After staining the cells, the Oil Red O solution was removed and the cells were washed three times with distilled water. For quantitative analysis under a microscope, cells stained with Oil Red O were observed by eluting with 100% isopropanol and measuring absorbance at 510 nm.

### Experimental Example 5. Quantitative Real-Time PCR

Quantitative real-time PCR (qRT-PCR) was performed to compare the expression of adipogenic regulators. Total RNA was extracted using a Hybrid-R RNA isolation kit (GeneAll, South Korea). The quantity and purity of the isolated total RNA were evaluated using a NanoDrop Lite spectrophotometer (Thermo Scientific, USA). Total RNA was used to synthesize first-strand complementary DNA using PrimeScript first-strand cDNA.

Synthesis kit (Takara, Japan) qRT-PCR was performed using TB Green Premix Ex Taq (Takara, Japan) in a Thermal Cycler Dice (TP700/760, using Takara β-actin as a standard).

Gene primers used for comparing genes and expression levels were listed in Table 1. Relative gene expression was analyzed in Thermal Cycler Dice (V5.0x; Takara, Japan) according to a 2-^{ΔΔCt} method.

TABLE 1

| Gene | | Sense | Oligonucleotide Sequence |
|---|---|---|---|
| Reference gene | β-actin | F | [symbol] |
| | | R | [symbol] |
| [symbol] | PPARγ | F | [symbol] |
| | | R | [symbol] |
| | [symbol] | F | [symbol] |
| | | R | [symbol] |
| | [symbol] | F | [symbol] |
| | | R | [symbol] |
| | FAS | F | [symbol] |
| | | R | [symbol] |
| | [symbol] | F | [symbol] |
| | | R | [symbol] |
| | [symbol] | F | [symbol] |
| | | R | [symbol] |
| | [symbol] | F | [symbol] |
| | | R | [symbol] |
| Cell cycle | CDK2 | F | [symbol] |
| [symbol] | | R | [symbol] |
| | [symbol] | F | [symbol] |
| | | R | [symbol] |
| | Cyclin A | F | [symbol] |
| | | R | [symbol] |
| | Cyclin D | F | [symbol] |
| | | R | [symbol] |

[symbol]

indicates data missing or illegible when filed

### Experimental Example 6. Animals, Diets, Experimental Design

All animal procedures were performed in accordance with the National Research Council guideline (Guide for the Care and Use of Laboratory Animals) and approved by the PuKyong National University Institutional Animal Care and Use Committee (PKNUJACUC-2021-14). Male C57BL/6 mice (6 weeks old, SamTako BioKorea, Osan, Korea) were accommodated in a temperature and humidity-controlled facility (22±2° C., 50%±10% relative humidity) so as to freely approach food and water on a 12 h/12 h light/dark cycle. After 7 days of adaptation, 24 mice were randomly divided into 4 groups (n=6) according to a diet type. A normal fat diet group (NFD; control group) fed a standard diet (D12450B 10% kcal of fat, Research Diets, USA). An obese model group fed a high-fat diet (HFD) (rodent diet containing 60% kcal fat) with or without treatment. The mice were orally treated with 200 μL of PBS or a daily dose of γ-PGA for 8 weeks. The animal's body weight was measured once a week at a predetermined time. Food intake was measured once every two days (FIG. 1B).

### Experimental Example 7. Glucose Tolerance Test

A glucose tolerance test was performed on mice fasted overnight on week 7 of the diet. A 50% glucose solution (1.5 g/kg body weight) was injected intraperitoneally, and blood was collected from the tail vein at 0, 15, 30, 60, 90, and 120 minutes after blood injection. Blood glucose was measured using a portable blood glucose meter (Accu-Chek Performa, Roche, Switzerland).

### Experimental Example 8. Evaluation of Homeostatic Model of Insulin Resistance

Homeostasis model evaluation of an insulin resistance (HOMA-IR) level was calculated using the following Formula:

### [Insulin Resistance Formula]

$$HOMA - IR = fasting plasma glucose ( mmol / L ) \times fasting plasma insulin ( mU / L ) / 22.5 .$$

Fasting blood glucose and fasting plasma insulin were measured using an automated chemistry analyzer (Accu-Chek, Roche Diabetes) and ELISA kit (Cat. No.: ab277390, Abcam, UK) according to the manufacturer's instructions.

### Experimental Example 9. Histological Analysis

Epididymal fat tissue was fixed with 4% paraformaldehyde, embedded in paraffin, sectioned (4 μm), and then stained with hematoxylin and eosin. Adipocyte size was measured and analyzed using Image-Pro (version 10.0.11, Media Cybernetics, USA). The liver lipid content of liver frozen sections was analyzed using Oil Red O staining and counterstained with hematoxylin.

As a result, the area stained red with Oil Red O was calculated as a total area of a cross section and expressed as a lipid area (% of total area).

### Experimental Example 10. Gut Microbiome Analysis

Total bacterial DNA was extracted from stool samples of mice at week 8 of the experiment using a QIAamp DNA Stool Mini kit (Qiagen, USA) according to a manufacturer's protocol. The amplified V3-V4 regions were sequenced using a forward primer (TCGTCGGCAG CGTCA GATGT GTATA AGAGA CAGCC TACGG GNGGC WGCAG) and a reverse primer (GTCTC GTGGG CTCGG AGATG TGTAT AAGAG ACAGG ACTAC HVGGG TATCT AATCC) and used for gut microbiome analysis.

### Experimental Example 11. Statistical Analysis

Data were statistically analyzed using one-way ANOVA using the Statistical Package for Social Sciences (SPSS; IBM, USA) and a Duncan's multiple range test was performed.

Data with P<0.05 were considered significant.

### Example 1. Separation and Characteristics of Two Types of γ-PGA

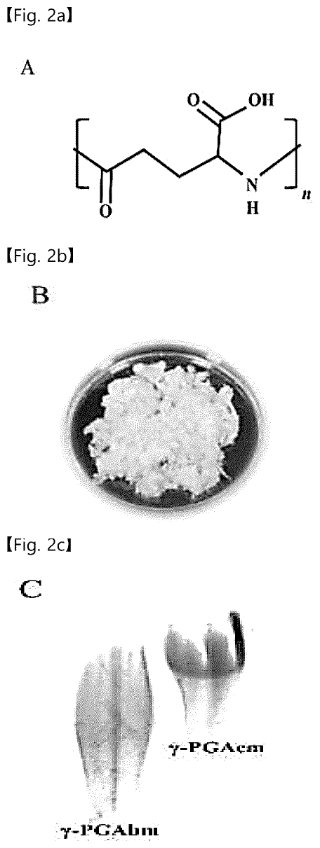

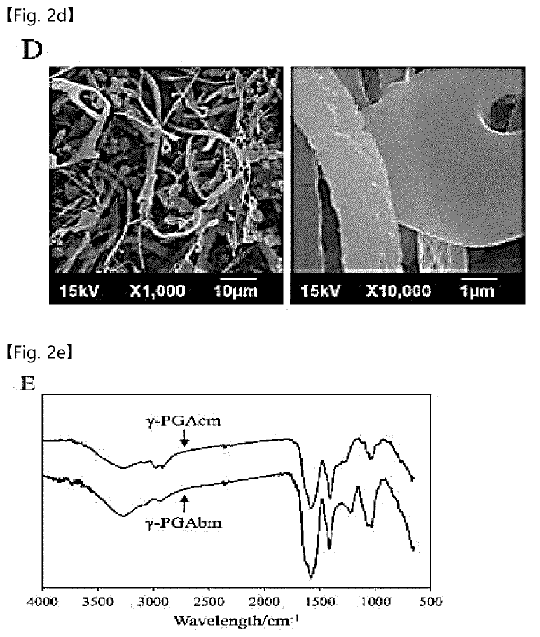

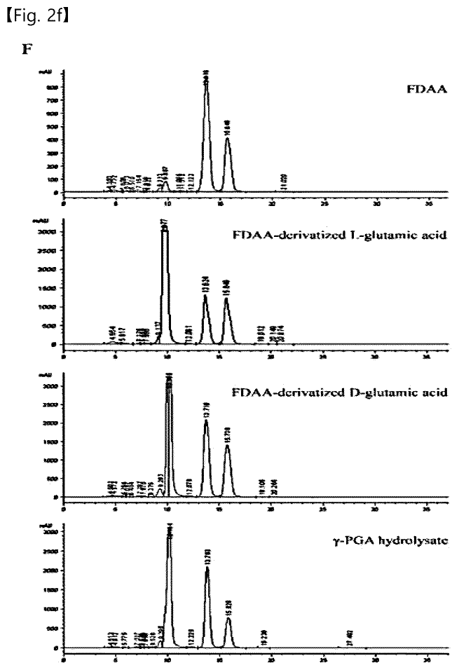

A chemical structure (FIG. 2A) and a final product (FIG. 2B) of γ-PGA were confirmed, and γ-PGA was divided into two types of γ-PGAbm (50 kDa to 400 kDa) and γ-PGAcm (400 kDa) with different molecular weights depending on a molecular weight composition. Through SDS-PAGE analysis, it was confirmed that γ-PGAbm showed a pattern with a wide molecular weight, and γ-PGAcm showed a pattern of γ-PGA with a relatively constant molecular weight (FIG. 2C).

As a result of analyzing γ-PGAbm and γ-PGAcm using a low vacuum scanning electron microscope (LV-SEM) and a small FT-IR analyzer (FT-IR) machine, it was confirmed that γ-PGAbm and γ-PGAcm, which were divided types of γ-PGA, had no significant differences in images and characteristics and shared the characteristics of general γ-PGA (FIGS. 2D and 2E). In addition, through HPLC analysis performed to analyze the enantiomeric composition of amino acids, it was confirmed that γ-PGAbm and γ-PGAcm were similar to γ-PGA, which was mostly composed of D-glutamic acid residues (FIG. 2F).

### Example 2. Toxicity Evaluation of γ-PGAbm and γ-PGAcm in Adipocytes

In order to confirm that γ-PGAbm and γ-PGAcm suppress adipogenesis, 3T3-L1 cells, which were preadipocytes, were treated with γ-PGAbm and γ-PGAcm for each concentration and cytotoxicity was confirmed through MTT analysis.

γ-PGAbm and γ-PGAcm confirmed the viability of a 3T3-L1 cell line at a concentration of 0.01%, and did not induce cytotoxicity, but it was confirmed that cytotoxicity to cells occurred at concentrations of 0.05% or higher of γ-PGAbm and γ-PGAcm.

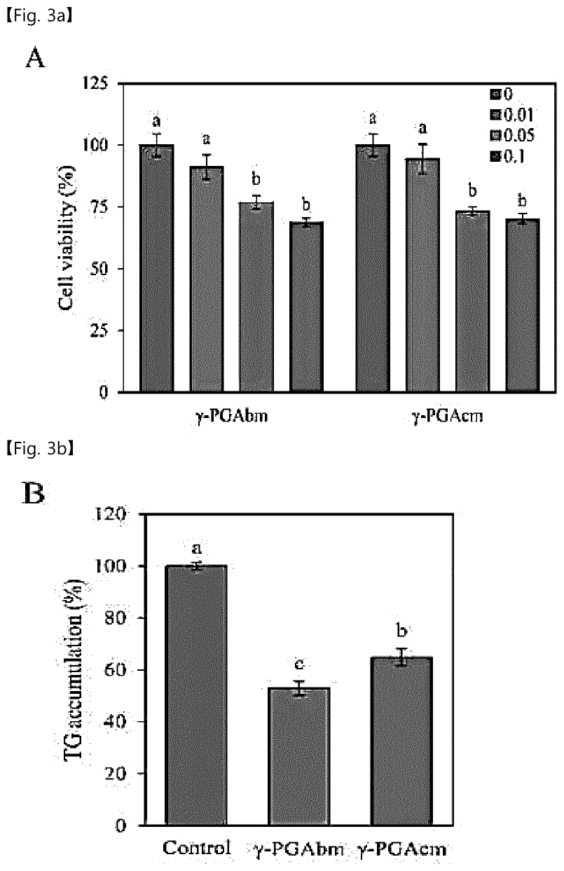

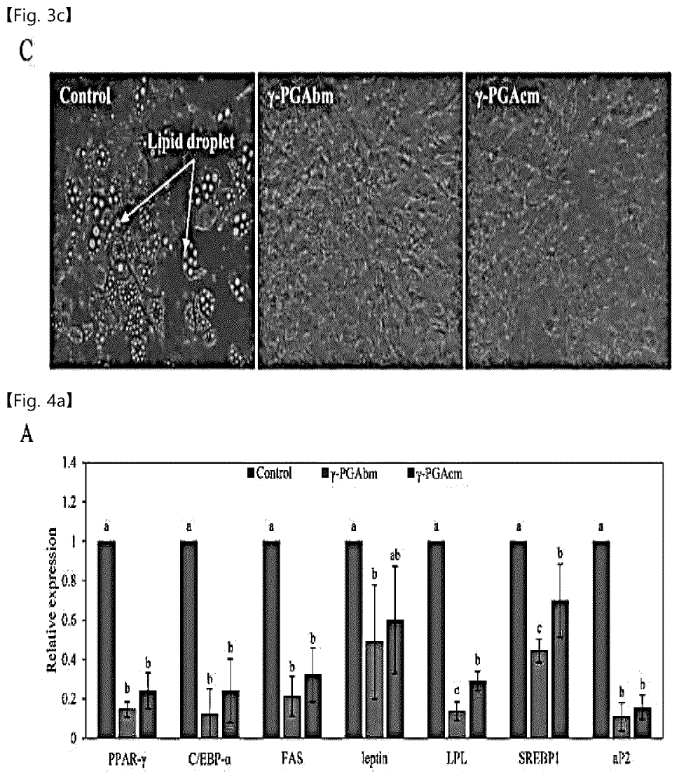

Accordingly, for the following experiments, γ-PGAbm and γ-PGAcm were used at concentrations of 0.01% or less (FIG. 3A).

### Example 3. Adipocyte Differentiation Inhibition Effect of γ-PGAbm and γ-PGAcm

In order to confirm that γ-PGAmb and γ-PGAcm inhibited adipocyte differentiation, a 3T3-L1 cell line, a preadipocyte cell line, was treated with γ-PGAbm and γ-PGAcm, and accumulation of lipid droplets and triglycerides were stained with Oil Red O to confirm adipocyte differentiation.

As a result of treating the 3T3-L1 cell line with γ-PGAbm and γ-PGAcm, and comparing the triglyceride contents quantitatively by staining with Oil Red O, it was confirmed that compared to untreated cells (control group) of the 3T3-L1 cell line, when treated with γ-PGAbm, the triglyceride content was decreased by 47.10%±2.73%, and when treated with γ-PGAcm, the triglyceride content was decreased by 35.09%±3.40% (FIG. 3B).

When the 3T3-L1 cell line was differentiated without treatment with γ-PGAbm and γ-PGAcm, it was confirmed that lipid droplets were significantly accumulated in the cells, and lipid droplet formation was inhibited upon treatment with γ-PGAbm and γ-PGAcm.

In particular, it was confirmed that the lipid droplet formation was significantly inhibited in γ-PGAbm-treated cells (FIG. 3C).

### Example 4. Expression of Genes Related to Adipogenic Markers in γ-PGAbm and γ-PGAcm

In order to confirm an effect of γ-PGAbm and γ-PGAcm on the expression of genes related to adipogenic markers, when the 3T3-L1 cell line, the preadipocyte cell line, was treated with γ-PGAbm and γ-PGAcm, the inhibition degrees of the expression of adipogenic transcription factors PPARγ, C/EBPα, FAS, LPL, SREBP1, aP2, and leptin were observed through RT-qPCR.

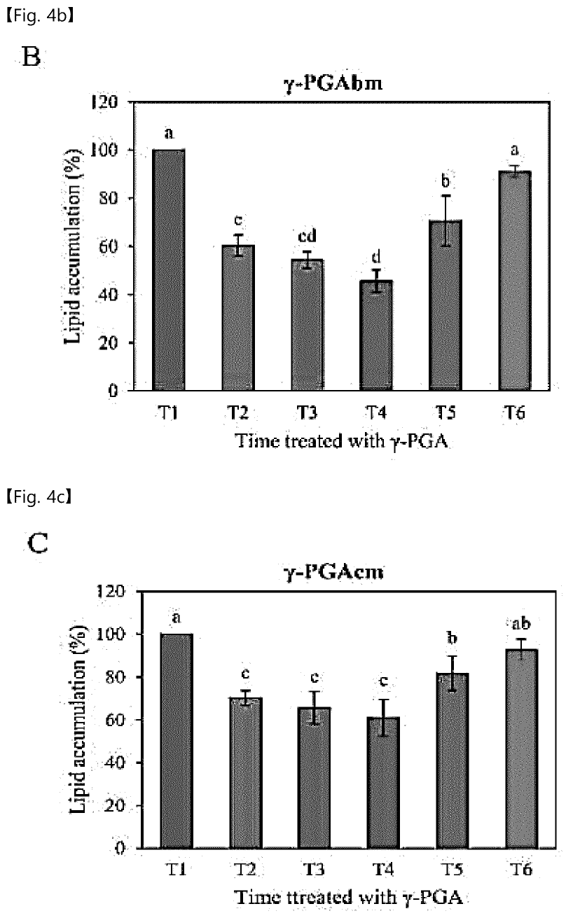

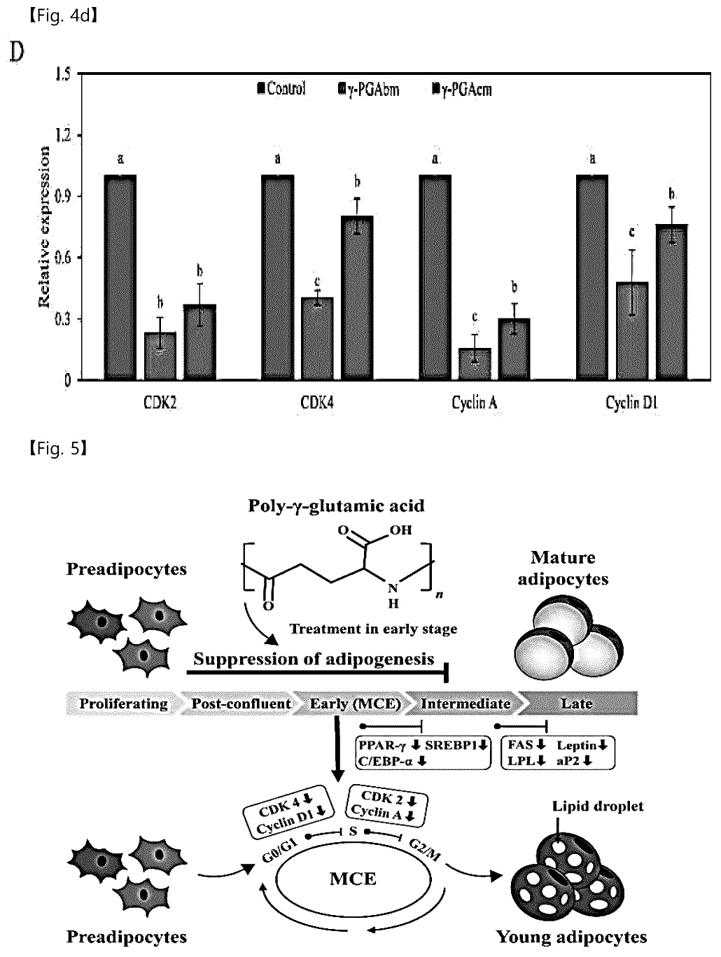

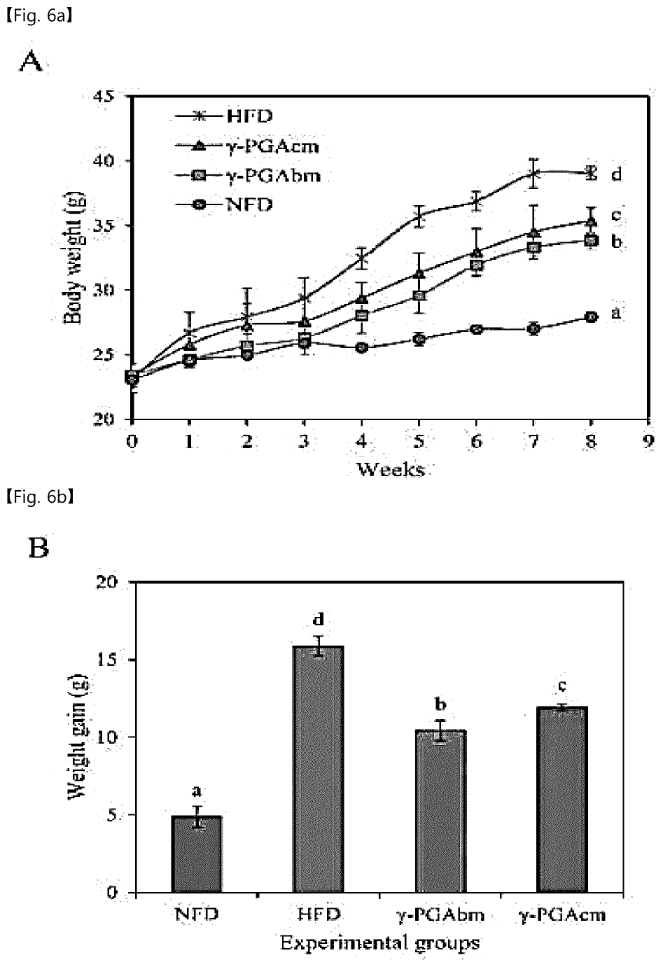

As a result, the genes related to adipogenic markers were PPARγ, C/EBPα, FAS, LPL and aP2, leptin, and SREBP1, and the expression thereof was all suppressed and reduced by the γ-PGAbm and γ-PGAcm treated groups (FIG. 4A).

In particular, the γ-PGAbm and γ-PGAcm groups showed significant differences in lipoprotein lipase (LPL) and sterol regulatory element binding protein 1 (SREBP1) (FIG. 4A).

### Example 5. Effect of γ-PGAbm and γ-PGAcm on Early Stage of Adipocyte Differentiation

To determine the effect of γ-PGAbm and γ-PGAcm on the early stage of adipocyte differentiation, when a 3T3-L1 cell line, which was a preadipocyte cell line, was treated with γ-PGAbm and γ-PGAcm, the differentiation inhibition stage was evaluated by dividing an adipogenic inhibition differentiation stage into early (day 0-2), intermediate (day 2-4), and late (day 4).

To evaluate at each stage of differentiation, the 3T3-L1 cell line was treated with γ-PGAcm and γ-PGAbm at a predetermined time for each stage, and accumulated intracellular lipids were stained with Oil Red O to confirm adipogenesis, and a high lipid inhibition effect was confirmed in the early stage of γ-PGAcm and γ-PGAbm (FIGS. 4B and 4C).

Therefore, it was suggested that γ-PGAcm and γ-PGAbm were involved in the early differentiation stage of adipocytes.

### Example 6. Expression of Genes Related with Cell Cycle Regulation in γ-PGAbm and γ-PGAcm

In order to confirm whether γ-PGAbm and γ-PGAcm were related with cell cycle regulation, when treating γ-PGAbm and γ-PGAcm in a mitotic clonal expansion (MCE) stage as a process of inducing adipocyte differentiation, the effect on the expression of genes affecting the cell cycle was examined.

The expression levels of Cyclin-dependent kinases (CDK) 2, CDK4, cyclin A, and cyclin D, which were genes related with cell cycle regulation, were significantly decreased by treatment with γ-PGAbm and γ-PGAcm compared to the control group. In particular, it was confirmed that the expression levels were effectively reduced in the γ-PGAbm group (FIG. 4D).

In addition, as shown in FIG. 5, it was confirmed that γ-PGA inhibited MCE in the early stage of adipocyte differentiation by regulating peroxisome proliferator-activated receptor gamma (PPAR-γ), sterol regulatory element binding protein 1 (SREBP1), and CCAAT/enhancer-binding protein alpha (C/EBP-α), and was involved in mitotic clonal expansion (MCE) and adipocyte differentiation by inhibiting the proliferation of 3T3-L1 preadipocytes.

Accordingly, it was suggested that the G0/G1 phase of the cell cycle was involved with CCAAT/enhancer-binding protein alpha (C/EBP-α), fatty acid synthase (FAS), leptin, lipoprotein lipase (LPL), adipocyte protein 2 (aP2), and cell cycle proteins, such as cyclin-dependent kinases (CDK) 4, cyclin D1, cyclin-dependent kinase 2 (CDK 2), and cyclin A (FIG. 5).

### Example 7. Obesity Therapeutic Effects of γ-PGAbm and γ-PGAcm in Animal Model

To confirm the obesity therapeutic effects of γ-PGAbm and γ-PGAcm, γ-PGAbm and γ-PGAcm were supplemented in an obesity-induced mouse model to confirm the effects related to obesity treatment.

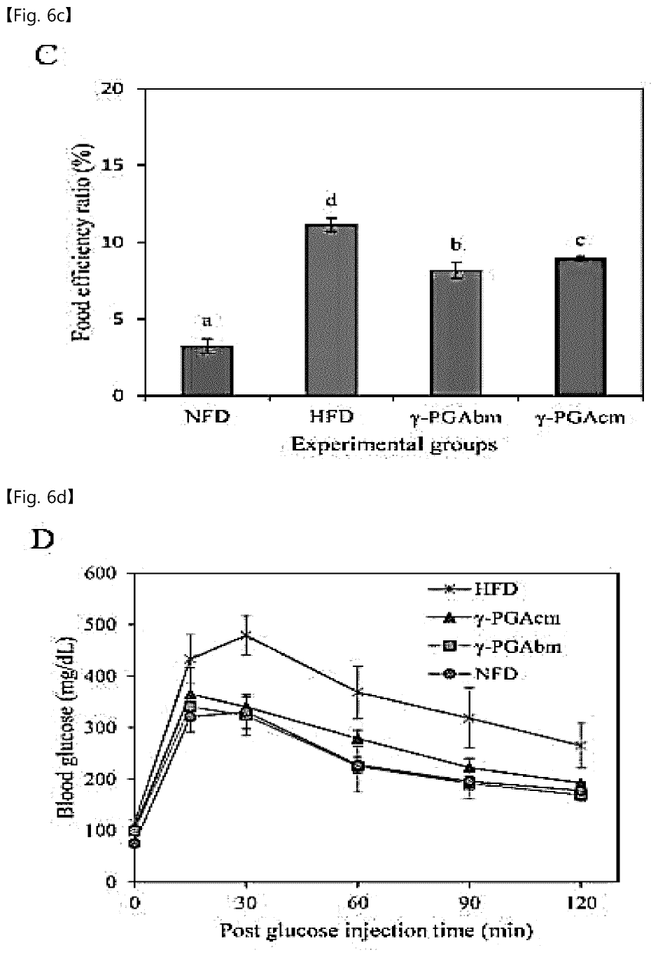

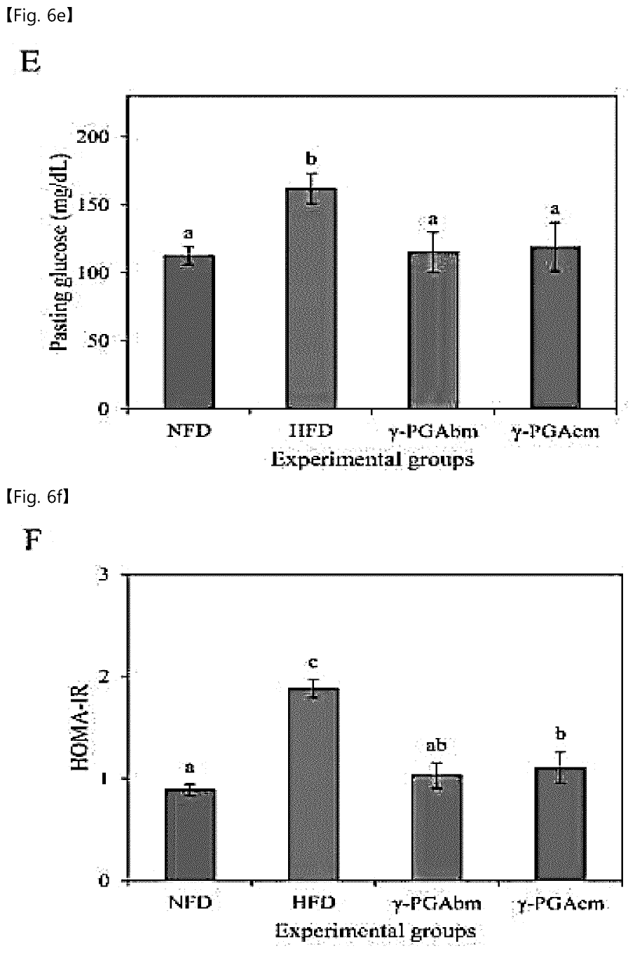

For 8 weeks, it was confirmed that a high-fat diet (HFD) group was increased in body weight, weight gain, and diet efficiency ratio compared to a normal diet (NFD) group (FIGS. 6A, 6B, and 6C).

For 8 weeks, the weight gain of the normal diet (NFD) group and the high-fat diet (HFD) group was increased to 4.88±0.70 and 15.87±0.62 g, respectively, but when the high-fat diet (HFD) group was treated with γ-PGAbm and γ-PGAcm, the weight gain was confirmed to 10.43±0.65 and 11.93±0.23 g, respectively. It was confirmed to be significantly reduced compared to a high-fat diet (HFD)-alone administered group (FIGS. 6B and 6C).

In addition, it was confirmed that the blood glucose level of the high-fat diet (HFD) group was significantly higher than those of other groups, and the blood glucose levels of the γ-PGAbm and γ-PGAcm groups in the high-fat diet (HFD) group were decreased compared to the high-fat diet (HFD) group (FIG. 6D).

In addition, it was confirmed that in the fasting blood glucose levels measured at 1 week before sacrificing mice, the fasting blood glucose level was lower in the γ-PGAbm and γ-PGAcm-supplemented groups in the high-fat diet (HFD) group than in the high-fat diet (HFD) group (FIG. 6E).

Therefore, it is suggested that γ-PGA affects a glucose tolerance test through a positive effect on glucose homeostasis.

### Example 8. Effect of γ-PGAbm and γ-PGAcm on Insulin Resistance (HOMA-IR)

To determine the effect of γ-PGAbm and γ-PGAcm on insulin resistance (HOMA-IR), insulin resistance (HOMA-IR) was compared in the normal diet (NFD) group, the high-fat diet (HFD) group, and in the γ-PGAbm and γ-PGAcm-supplemented groups in the high-fat diet (HFD) group in the obesity-induced mouse model.

The insulin resistance (HOMA-IR) indexes of the normal diet (NFD) group, the high-fat diet (HFD) group, and the γ-PGAbm and γ-PGAcm-supplemented groups in the high-fat diet (HFD) group were 0.89±0.05, 1.88±0.09, 1.03±0.12, and 1.11±0.15, respectively. It was confirmed that the γ-PGAbm and γ-PGAcm-supplemented groups in the high-fat diet (HFD) group reduced the insulin resistance (HOMA-IR) as compared to the high-fat diet (HFD) alone group and thus, γ-PGAbm and γ-PGAcm improved the insulin resistance (HOMA-IR) (FIG. 6F).

### Example 9. Histological Effects of γ-PGAbm and γ-PGAcm

In order to confirm the histological effects of γ-PGAbm and γ-PGAcm, in the normal diet (NFD) group, the high-fat diet (HFD) group, and the γ-PGAbm and γ-PGAcm-supplemented groups in the high-fat diet (HFD) group in the obesity-induced mouse model, morphological characteristics of mouse epididymal white adipose tissue sections were confirmed by Oil Red O staining.

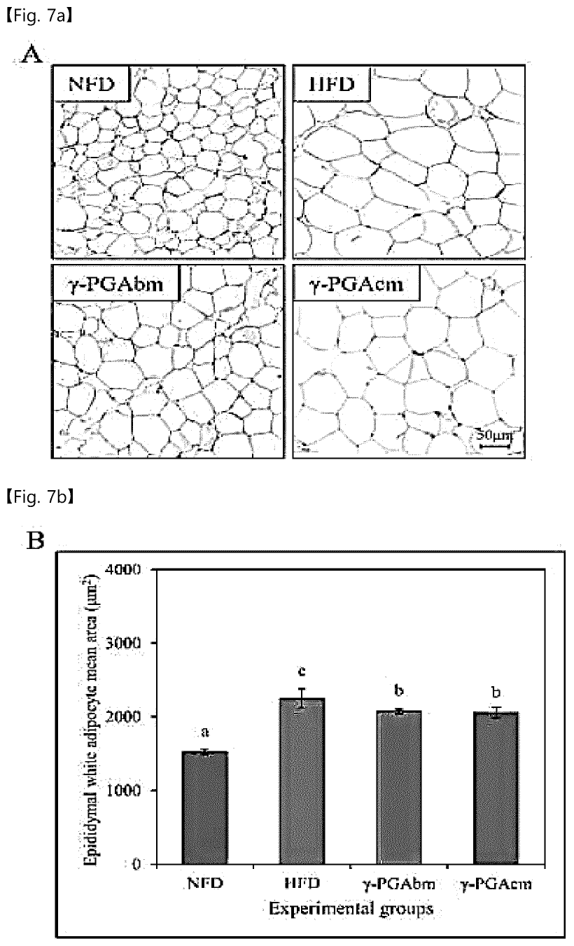

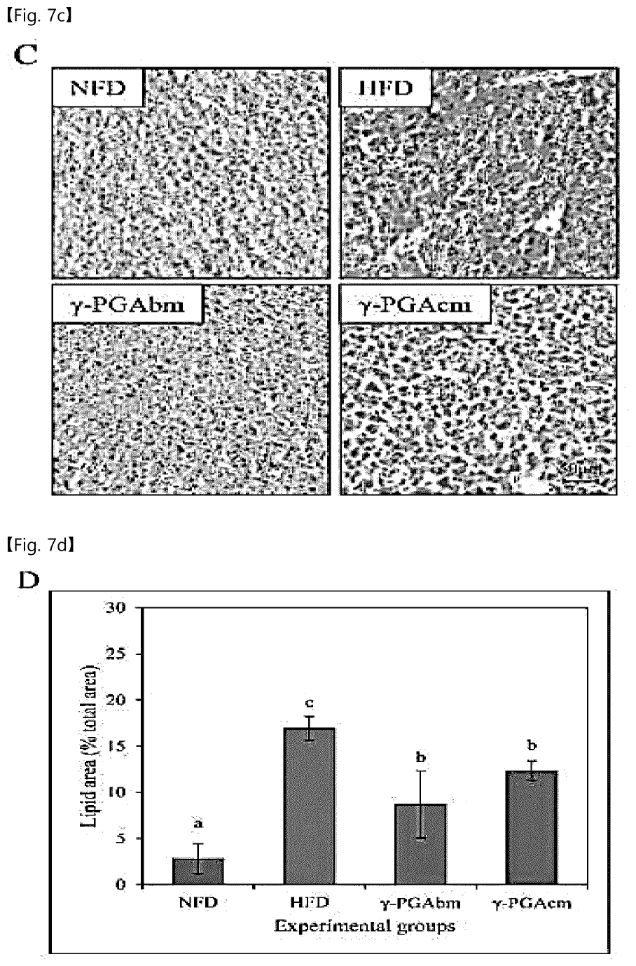

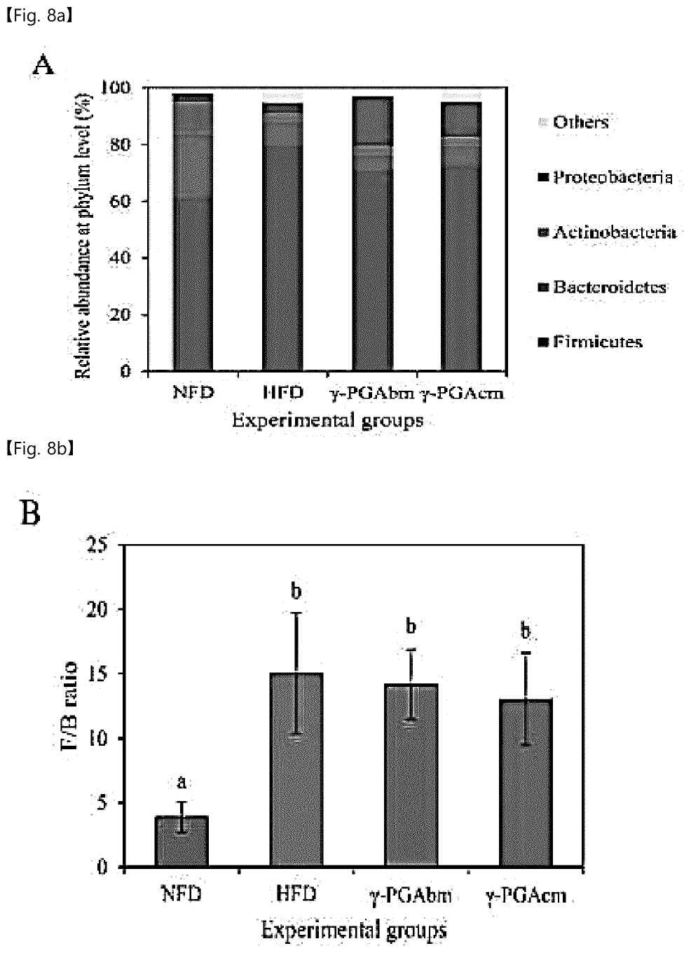

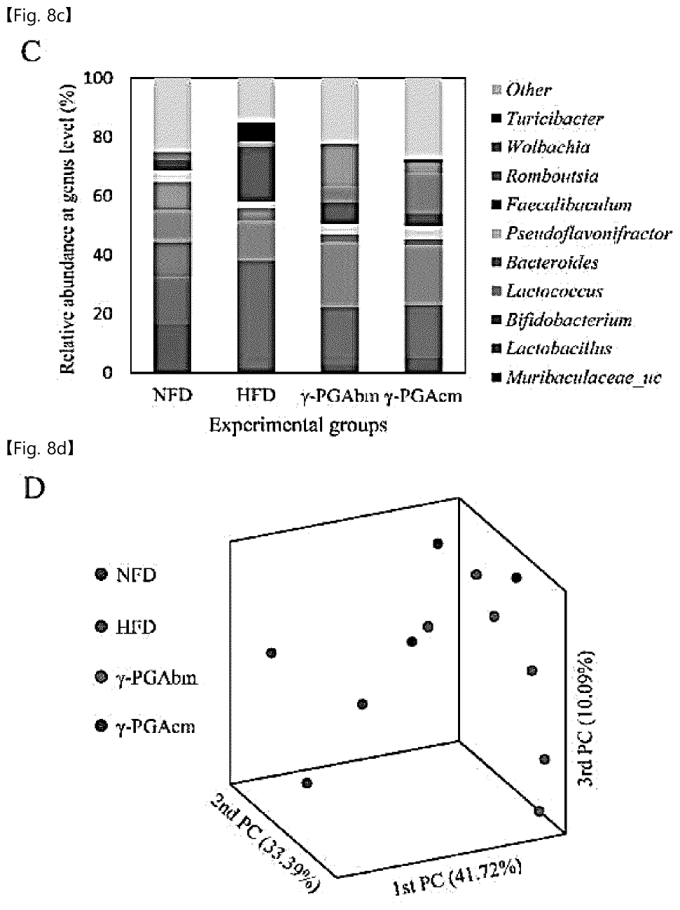

It was confirmed that in the high-fat diet (HFD) group, the tissue was obese compared to the normal diet (NFD) group (FIG. 7A). In addition, it was confirmed that in the γ-PGAbm and γ-PGAcm supplemented groups, the average area of the epididymis in the white adipose tissue was improved compared to the high-fat diet (HFD) group (FIG. 7B).

It was confirmed that the lipid content of the liver analyzed by Oil Red O staining showed a similar trend to that of epididymal white adipose tissue, the high-fat diet (HFD) group had the highest lipid area ratio of 16.91%±1.29%, the γ-PGAbm treated group had the lipid area ratio of 8.66%±3.64%, and the γ-PGAcm treated group had the lipid area ratio of 12.31%±1.05%, which was improved compared to the high-fat diet (HFD) (FIGS. 7C and 7D).

### Example 10. Gut Microbiome Analysis

There was no significant difference in α-diversity abundance according to OTU, ACE, and Chao1 indices in the γ-PGAbm and γ-PGAcm-supplemented groups, and there was a significant difference in estimates for Shannon and Simpson (Table 2).

TABLE 2

| Experimental | | | | | |
|---|---|---|---|---|---|
| groups | OTUs | ACE | Chao1 | Shannon | Simpson |
| NFD | 516.33 ± 55.77 | 596.77 ± 47.02 | 599.57 ± 40.31 | 3.85 ± 0.34 | 0.06 ± 0.02 |
| HFD | 468.33 ± 56.16 | 562.63 ± 64.16 | 562.31 ± 77.68 | 3.49 ± 0.21 | 0.07 ± 0.01 |
| γ-PGAbm | 479.00 ± 54.34 | 554.20 ± 64.25 | 561.59 ± 64.47 | 3.34 ± 0.35 | 0.11 ± 0.06 |
| γ-PGAcm | 452.33 ± 72.86 | 530.93 ± 100.42 | 529.93 ± 104.80 | 3.45 ± 0.45 | 0.09 ± 0.04 |

Firmicutes were the most abundant in all groups, but the ratio was varied every group. The normal diet (NFD) group had the lowest at 60.83%, and the high-fat diet (HFD) group had the highest at 79.41%.

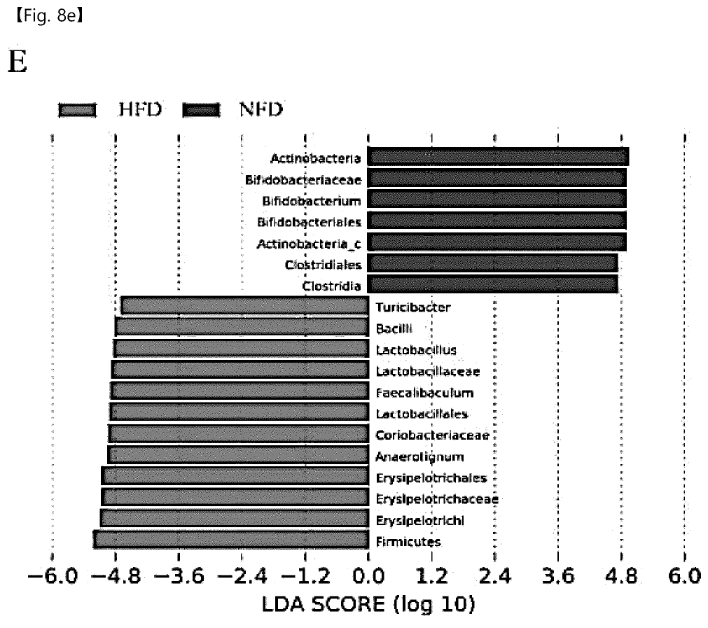

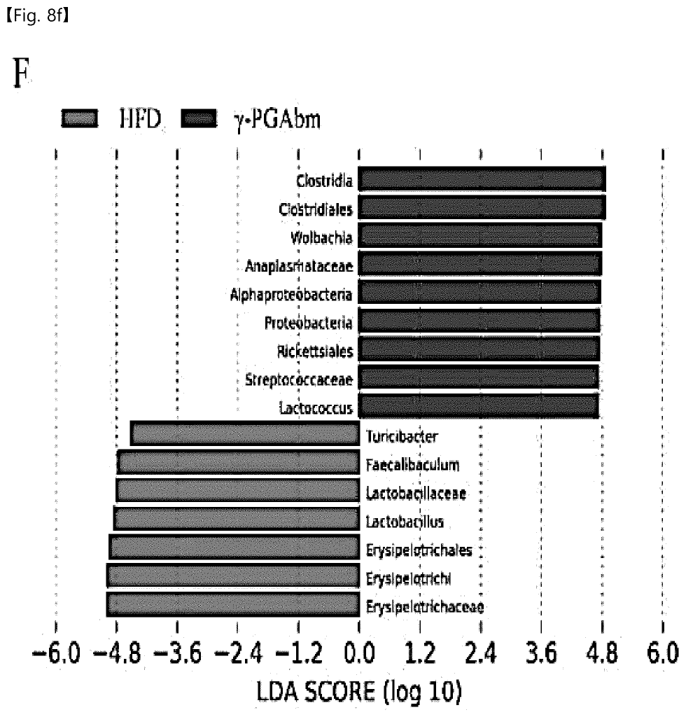

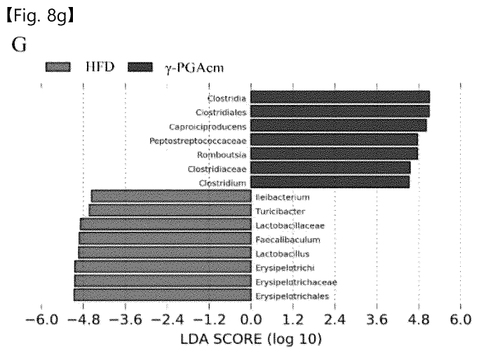

The γ-PGAbm and γ-PGAcm groups were confirmed to be 70.46 and 72.05%, respectively (FIG. 8A). It was confirmed that the Firmicutes/Bacteroidetes ratio was lowest in the normal diet (NFD) group (FIG. 8B), and Lactobacillus, Faecalibaculum, and Turicibacter at the genus level were most abundant in the high-fat diet (HFD) group (FIG. 8C).

In beta diversity analyzed based on a UniFrac metric through principal coordinate analysis (PCoA), distinct clustering was confirmed among the normal diet (NFD) group, the high-fat diet (HFD) group, and the γ-PGAbm and γ-PGAcm-supplemented groups in the high-fat diet (HFD) group.

On the other hand, it was confirmed that there was no significant difference in clusters in the γ-PGAbm and γ-PGAcm-supplemented groups in the high-fat diet (HFD) group (FIG. 8D). In analysis of specific bacterial taxa through linear discriminant analysis effect size (LEfSe), it was confirmed that Erysipelotrichia, Erysipelotrichales, Erysipelotrichaceae, Faecalibaculum, Turicibacter, Lactobacillaceae, and Lactobacillus were more abundant in the high-fat diet (HFD) group than in the normal diet (NFD) group and the γ-PGAbm and γ-PGAcm-supplemented groups in the high-fat diet (HFD) group (FIGS. 8E, 8F, and 8G).

Hereinabove, the present disclosure has been described with reference to preferred exemplary embodiments thereof. It will be understood to those skilled in the art that the present disclosure may be implemented as a modified form without departing from an essential characteristic of the present disclosure. Therefore, the disclosed exemplary embodiments should be considered in an illustrative viewpoint rather than a restrictive viewpoint. The scope of the present disclosure is illustrated by the appended claims rather than by the foregoing description, and all differences within the scope of equivalents thereof should be construed as being included in the present disclosure.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

What is claimed is:

1. A method for treating or improving obesity, comprising administering to a subject in need thereof a composition comprising poly-γ-glutamic acid (γ-PGA) as an active ingredient.

2. The method of claim 1, wherein the poly-γ-glutamic acid (γ-PGA) is γ-PGAbm or γ-PGAcm.

3. The method of claim 2, wherein the γ-PGAbm or γ-PGAcm is isolated from Bacillus sp. SJ-10 according to a molecular weight composition.

4. The method of claim 3, wherein the molecular weight is 50 kDa to 400 kDa for γ-PGAbm and 400 kDa for γ-PGAcm.

5. The method of claim 1, wherein the subject requires suppressing differentiation of adipocytes.

6. The method of claim 5, wherein the subject requires suppressing adipocyte differentiation in relation to the early stage of adipocyte differentiation.

7. The method of claim 1, wherein the subject requires suppressing cell cycle regulatory genes, Cyclin-dependent kinases (CDK) 2, CDK4, cyclin A, and cyclin D.

8. The method of claim 1, wherein the subject requires suppressing the expression of adipogenesis-related genes.

9. The method of claim 8, wherein the adipogenesis-related genes are peroxisome proliferator-activated receptor gamma (PPARγ), CCAAT-enhancer-binding proteins α (C/EBPα), FAS, lipoprotein lipase (LPL), Sterol regulatory element-binding protein 1 (SREBP1), activating protein-2 (aP2), and leptin.

10. The method of claim 1, wherein the subject requires reducing body weight, weight gain, diet efficiency and blood glucose level.

11. The method of claim 1, wherein the subject requires reducing insulin resistance and lipid content.

12. The method of claim 1, wherein the subject requires regulating gut microorganisms.

13. The method of claim 12, wherein the gut microorganisms increase a ratio of Firmictes/Bacteroides.

14. The method of claim 12, wherein when γ-PGABM is treated in the gut microorganisms, Clostridia, Clostridiales, Wolbachia, Anaplasmataceae, Alphaproteobacteria, Rickettsiales, Streptococcacceae, and Lactococcus are increased, and Erysipelotrichia, Erysipelotrichales, Erysipelotrichaceae, Faecalibaculum, Turicibacter, Lactobacillaceae, and Lactobacillus are decreased, thereby controlling obesity.

15. The method of claim 12, wherein when γ-PGAcm is treated in the gut microorganisms, Clostridia, Clostridiales, Caproiciproducens, Peptostreptococcaceae, Romboutsia, Clostridiaceae, and Clostridium are increased, and Ileibacterium, Turicibacter, Lactobacillaceae, Faecalibaculum, Lactobacillus, Erysipelotrichia, Erysipelotrichales, Erysipelotrichaceae, and Erysipelotrichaceae are decreased, thereby controlling obesity.

16. A method for regulating intestinal bacteria caused by obesity comprising administering to a subject in need thereof a composition comprising poly-γ-glutamic acid (γ-PGA) as an active ingredient.

17. The method of claim 16, wherein the subject requires increasing Clostridia, Clostridiales, Wolbachia, Anaplasmataceae, Alphaproteobacteria, Rickettsiales, Streptococcacceae, and Lactococcus, and decreasing Erysipelotrichia, Erysipelotrichales, Erysipelotrichaceae, Faecalibaculum, Turicibacter, Lactobacillaceae, and Lactobacillus, comprising treating poly-γ-glutamic acid (γ-PGA).

18. The method of claim 16 wherein the subject requires increasing Clostridia, Clostridiales, Caproiciproducens, Peptostreptococcaceae, Romboutsia, Clostridiaceae, and Clostridium, and decreasing Ileibacterium, Turicibacter, Lactobacillaceae, Faecalibaculum, Lactobacillus, Erysipelotrichia, Erysipelotrichales, Erysipelotrichaceae, and Erysipelotrichaceae, comprising treating poly-γ-glutamic acid (γ-PGA).
